(19) United States
(12) Patent Application Publication
(10) Pub. No.: US 2025/0277209 A1
(43) Pub. Date: Sep. 4, 2025

(54) MECHANISM-AGNOSTIC DIRECTED EVOLUTION OF PROTEIN THERAPEUTICS

(71) Applicant: William Marsh Rice University, Houston, TX (US)

(72) Inventors: Jerzy Olgierd Szablowski, Houston, TX (US); Nicolas Sebastian Buitrago, Houston, TX (US)

(21) Appl. No.: 19/066,979

(22) Filed: Feb. 28, 2025

Related U.S. Application Data
(60) Provisional application No. 63/559,995, filed on Mar. 1, 2024.

(51) Int. Cl. C12N 15/10

(52) U.S. Cl. CPC C12N 15/1082; C12N 15/1089

(57) ABSTRACT

In accordance with this approach genetically-encodable proteins and peptides are used as drugs in certain embodiments. A gene delivery is performed into a region of diseased tissue. A different peptide or protein is then expressed in each cell. After a period of expression the site of injection is biopsied and the phenotype of cells analyzed for presence of disease or other traits of interest. The cells showing a positive resolution or mitigation of the disease or exhibiting the trait of interest are then analyzed to identify the DNA that led to a peptide/protein that mitigated the disease or exhibited the trait of interest.

### CROSS-REFERENCE TO RELATED APPLICATIONS

This application claims priority to U.S. Provisional Application No. 63/559,995, entitled “Mechanism-Agnostic Directed Evolution Of Protein Therapeutics”, filed Mar. 1, 2024, which is incorporated by reference in all its entirety herein.

### STATEMENT OF GOVERNMENT SUPPORT

This invention was made with government support under W911NF-23-1-0017 awarded by the Army Research Laboratory-Army Research Office. The government has certain rights in the invention.

### TECHNICAL FIELD

The subject matter disclosed herein relates to the field of phenotypic drug discovery.

### BACKGROUND

Modern drug discovery heavily relies on identifying specific disease targets or mechanisms, which often require significant time and resources. This is often a rate-limiting step in developing therapeutics for complex diseases such as nonalcoholic fatty liver disease and nonalcoholic steatohepatitis (NASH). In some cases, despite identifying potential targets, drugs may still fail to show efficacy in the clinic.

In particular, the current paradigm in drug development is to find a therapeutic ‘target’ and attempt to make a drug that has a high potency and high affinity to a pre-identified cell-receptor. The drug is then developed to bind to that target with specificity in an in vitro setting. Some of the drugs are then deemed as promising by virtue of their binding affinity, signaling action in cells, and computational analysis of probable toxicity and pharmacokinetics in vivo. A focused library of compounds is then tested in vivo in animals. Many of these in vitro-identified compounds have a subpar performance and are further optimized in small animals, before being advanced to larger animal trials. The process of pre-clinical research, where drugs are being selected in vitro before being advanced to in vivo testing is time consuming, expensive, and relatively low-yield. For example, in the central-nervous system, less than 10% of drugs identified through this process succeed in clinical trials. Even if successful, the drugs that result from this development pipeline often have significant side effects and do not translate well between animal species, or between the animal and clinical trials, leading to frequent failure of translation of these therapeutics at each step of the development.

In short, drug development and many other fields of bioscience face a problem-they require understanding of the disease pathophysiology on a molecular level within the living organisms. Not all of these interactions are known, testing against all of them in vivo using the current therapeutic development paradigm would accrue extremely high cost, and the molecular basis of disease is usually too poorly understood and/or too complex to be solved with computational design.

### BRIEF DESCRIPTION

An alternative drug design pipeline is disclosed herein for optimizing the drugs for in vivo performance without an a priori assumption of the mechanism. Instead, we look for drugs which are most effective and safe in vivo from the start. This method is referred to herein as Mechanism Agnostic Peptide Screen (MAPS).

In accordance with this approach, genetically-encodable proteins and peptides are used as drugs in certain embodiments. A gene delivery is performed into a region of diseased tissue. A different peptide or protein is then expressed in each cell. In certain embodiments more than one peptide or protein can also be expressed in each cell to allow for observing effects of drug combinations on the cells. After a period of expression the site of injection is biopsied, and analyzed for the phenotype of cells for presence or lack of disease. In accordance with this approach, such phenotypes can include, but are not limited to, visual appearance under microscope, protein expression levels, changes in gene expression, metabolism, or any other change identifiable on single-cell level. The cells showing a positive resolution or mitigation of the disease are then analyzed to identify the DNA of MAPS peptides that encoded to a MAPS peptide/protein that mitigated the disease. The successful peptides/proteins are re-screened to confirm their performance as therapeutics. Once confirmed, the peptides/proteins are tested in low-throughput in an in vivo model by delivering them to the tissue or cells of interest.

In some cases the proteins/peptides are expressed intracellularly and the process screens for intracellular peptide drugs. In some cases the proteins/peptides are displayed extracellularly on the cell surface during the screen. The extracellular part of the peptide, or a part thereof, is then used as an extracellular drug interfacing with the cell surface. In some cases these proteins/peptides are produced in cells through gene therapy, in other cases they are delivered through intravenous, subcutaneous, intramuscular, or direct injection into the organ, or through oral, intradermal, inhalable, or other routes. In the case of intracellular proteins/peptides the delivery is intracellular, and in the case of extracellular receptors, the delivery of genes encoding the peptides can be intracellular, or to in the case of delivery of peptides identified with MAPS, to the interstitial space of the tissues containing the cells of interest either through a direct injection or through a systemic delivery. In some cases the protein/peptides are used to screen for cell survival. For example, in case of neurodegenerative diseases, or any other diseases resulting in cell loss, the cell survival after expression of the protein/peptide rather than phenotypic features of the cells can be used as a basis for considering the candidate drug successful. In some cases the cell phenotype will be evaluated using single-cell mRNA sequencing, in which case the mRNA encoding the protein/peptide will be used to identify the protein/peptide identity. In some cases the whole transcriptome will be used to evaluate which transcripts were changed by the cell, including the transcripts signifying cellular toxicity of the peptide. In some cases the recovery of cells containing expressed proteins/peptides can receive more than one copy of the DNA encoding the proteins/peptides. In some cases, different proteins/peptides will be encoded in the same cell. In such a form MAPS allows for screening for protein/peptide drug combinations.

Further, in some cases we may aid the screening process with machine learning. While high-throughput screening allows for the fitness characterization of thousands of molecules, it may be laborious, time-consuming, and resource intensive. To address this, in silico directed evolution may be employed to optimize the fitness of experimentally characterized molecules. In this cases, a “low-N” directed evolution paradigm may be used, where a machine-learning model is built hierarchically starting from global information of all functional proteins and peptides, then tuned with evolutionarily or significantly similar peptides to those that are experimentally characterized, and finally trained on mechanism agnostic peptide screening data.

In MAPS, many peptides with varied sequences will be tested. In some cases, this will result in a collection of characterized peptides with low sequence similarity, making sequence alignment and other analyses for in silico optimization challenging. For example, using vector-clustered multiple sequence alignment combined with the “low-N” approach in these cases would allow for optimized sequence homology searches. This would allow in silico directed evolution of a target class of peptides by only characterizing a small number of peptide candidates by experimental screening, further accelerating drug development. Advantages of the presently disclosed techniques include, but are not limited to: lower cost by bypassing in vitro testing and streamlining lead optimization (e.g., MAPS can test between 1 peptide and the number of peptides equivalent to the number of cells in the tested tissue).

In a first aspect, a method is provided for phenotype-driven drug discovery. In accordance with this method, a nucleic acid library is packaged in a vector to generate a vector library for gene delivery. The nucleic acid library encodes randomized peptides. The vector library is applied to a cellular model at a threshold multiplicity of infection corresponding to single vector transduction per cell. The transduced single cells are screened for cell survivability or one or more phenotypes to identify one or more peptides of interest. In one case, the vector library uses viral vectors, such as adeno-associated viral vectors (AAVs) or lentiviral vectors. In other cases, synthetic vectors, such as nanoparticles or liposomes can be used for construction of the library.

In a further aspect, an engineered extracellular protein carrier (EPC) is provided. In accordance with this aspect, the EPC comprises: a leader sequence for display of the EPC on a cell membrane, a receptor structure allowing for the anchoring of the EPC to the membrane, an optional linker to enable interactions of the therapeutic peptide within the same cells, and a therapeutic protein or peptide candidate that is displayed either intra- or extracellularly. Additionally, the EPC may contain a label allowing for facile detection of the EPC within the cell, such as an immunostaining tag or a fluorescent protein.

Once the peptide therapeutics are identified through the MAPS screen, either intracellularly with or without EPC, or extracellularly with EPC-based screen, the therapeutic peptides obtained through the screen can be used as a basis for generating systemically-administered peptide drugs. Any part of the EPC can serve as such drug, including the part designated herein as the “therapeutic peptide candidate” which was present within the library of peptides screened with MAPS. Such therapeutic peptide can be presented as gene therapy on the EPC or can be expressed and secreted from the cells. It can also be synthesized chemically or through recombinant protein production and administered without gene therapy as a protein/peptide therapeutic. The EPC drug can be further modified to improve its properties either through additional rounds of MAPS screening, or through chemical modifications.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1. Schematic of EPC design. Therapeutic peptides are tethered to cell membranes of genetically specified cells.

FIG. 2 Overview of EPC validation assay in an in vitro NASH model. Lipid accumulation is induced in HepG2 cells before treatment with EPCs containing sequences of therapeutic peptides. Lipids are quantified by flow cytometry or imaging using fluorescent lipid dyes.

FIG. 3 Preliminary validation of EPCs. Lipids are reduced in HepG2 cells after treatment with EPCs containing either poly-glycine (M(G)7) or AWRK6 (A6) peptide sequences, but not with empty EPC scaffold.

FIG. 4 For preliminary sorting of steatotic and wild-type HEPG2 cells, distribution of steatotic (mScarlet expressing) and wild type HepG2 cells after bulk separation by percoll gradient centrifugation.

FIG. 5 For preliminary sorting of steatotic and wild-type HEPG2 cells, mean±standard deviation of lipid fluorescence from separated cells in FIG. 4.

FIG. 6 For preliminary sorting of steatotic and wild-type HEPG2 cells, lipid distribution for cells separated by 90% percoll gradient.

FIG. 7 For preliminary sorting of steatotic and wild-type HEPG2 cells, fraction of L and H-HepG2 cells across different bins of the lipid distribution.

FIG. 8 Overview of mechanism agnostic peptide screening. Diverse DNA libraries encoding randomized peptides are packaged in viral vectors for efficient gene delivery. Viral vector libraries are applied to a NASH disease model at a low multiplicity of infection for single cell tethered peptide expression using EPCs. Cells are screened based on lipid accumulation and sequenced to recover peptide information.

FIG. 9 depicts an overview of in vivo validation of extracellular peptide carriers.

FIG. 10 graphically depicts results of the overview described with respect to FIG. 9.

FIG. 11 Principles and design of Mechanism Agnostic Therapeutic Screening (MAPS). Library of random peptides is expressed on the surface of BAT adipocytes. Mice are then challenged with cold; After 6 h their tissues are harvested and homogenized into single cells. Single-cell sequencing is used to measure thermogenic gene activity and link it to the RNA encoding the peptide that induced the thermogenesis. The top ˜10 most thermogenic peptides are further mutated and subjected to 3 more rounds of evolution

FIG. 12 Principles and design of Mechanism Agnostic Therapeutic Screening (MAPS). MAP peptides are evolved to act through extracellular interaction with receptors on BAT adipocytes. (The peptides are tethered to a transmembrane domain on a flexible linker to avoid steric hindrance). After screening, the extracellular portion becomes a systemically-injected drug optimized to act on BAT cells' surface.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and enterprise-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

As discussed herein, the presently described techniques relate to mechanism-agnostic, relatively inexpensive (i.e., “cheaper”) therapeutic development, including high-throughput screening and in vivo high-throughput screening for drugs, including peptide drugs. In certain contexts viral vectors may be involved.

Since the introduction of molecular biology, drug development has been primarily target driven. This approach generally involves identifying molecular targets that are implicated in disease processes. Once disease targets have been established, drug candidates are carefully designed and screened based on their ability to interact with these targets effectively and selectively. Promising candidates are evaluated and optimized further in preclinical testing, before proceeding to clinical trials. This target driven pipeline has yielded many effective therapeutics for a wide range of diseases. However, many challenges remain. First, significantly low success rates, sometimes below 20%, have plagued target driven drug discovery. Unfortunately, many of these failures are due to lack of efficacy, despite promising preclinical results. This discrepancy poses a significant hurdle in drug discovery and has caused many to reevaluate target driven approaches. Second, the time scale of developing a single new drug is on the order of decades, making it difficult to iterate quickly until a solution is found. Consequently, decades may be spent trying to find novel therapies with little success.

In short, modern drug discovery heavily relies on identifying specific disease targets or mechanisms, which often require significant time and resources. This is often a rate-limiting step in developing therapeutics for complex diseases such as nonalcoholic fatty liver disease and nonalcoholic steatohepatitis (NASH). In some cases, despite identifying potential targets, drugs may still fail to show efficacy in the clinic.

With this in mind, the current paradigm in drug development is to find a therapeutic ‘target’ (e.g., TP53) and attempt to make a drug that has a high potency and high affinity to a pre-identified cell-receptor. The drug is then developed to bind to that target with specificity in an in vitro setting. Some of the drugs are then deemed as ‘promising by virtue of their binding affinity, signaling action in cells, and computational analysis of probable toxicity and pharmacokinetics. A focused library of compounds is then tested in vivo in animals. Many of these in-vitro identified compounds often have a subpar performance and are further optimized in mice, before being advanced to larger animal trials. The process of pre-clinical research, where drugs are being selected in vitro before being advanced to in vivo testing is time consuming, expensive, and relatively low-yield. For example, in the central-nervous system, less than 10% of drugs succeed in clinical trials. Even if successful, the drugs that result from this development pipeline often have significant side effects and do not translate well between animal species, or between the animal and clinical trials.

There are many possible reasons for such failings:
a) Possible reason 1: In vitro testing is not a good predictor of an in vivo system—For example, we test binding to a receptor in HEK293 cells, but in a mouse there are thousands of different cell types that compete for drug interaction. There's a potential for immune action, blood vessels passage, kidney clearance . . . all these things are not recapitulated in vitro.
b) Possible reason 2: Assumption that binding to a single receptor is curative—There are many drugs that bind to a single receptor and have a therapeutic effect. However, biology works by interacting with multiple receptors at once. Screening multiple drugs at once would be very costly so it is not generally done. Nevertheless, there is no guarantee that a disease can be cured by an action of single receptor, since usually it an ensemble of many molecules that drives biological processes.
c) Possible reason 3: Assumption that specific mechanism of action is the same in vitro and in vivo—The specificity is often assumed to be well correlated with potency, since with few molecules in the body, one would expect there will be less non-specific binding events. This may not be universally true, because drugs developed using this paradigm have a slew of side effects.

Despite advances in every stage of the drug discovery process, frequent failures for diseases with known targets suggest that the method other than ones that are target-driven are needed.

As discussed herein, a technique is described to address current failing in the current target-driven pharmaceutical development approaches. In accordance with the presently contemplated approaches, and in the context of a Parkinson's disease (PD) context, we would deliver genes to the dopaminergic cells in a model of PD. These genes would encode short peptides, and small fraction of them would interface with receptors that counteract the mechanism of cell death in PD by pure chance. To improve our chances we would deliver very large numbers of those peptides, with each gene delivery virus only containing one type of peptide. We would dilute the dose of a viral vector so most cells would only receive one virus. Over time, the genes would produce peptides intracellularly. We would then extract the surviving dopaminergic cells from the brain and sequence the viral mRNA or DNA to obtain a list of peptide sequences that were present in surviving neurons. The list of DNA sequences would then be sent to twist biosciences to generate an oligopool, and re-screened in another mouse to confirm causality of the neuronal survival for each peptide. Finally, we would then perform low-throughput testing where top ˜5 drugs would be tested in multiple mice.

This approach can be generalized to many different diseases or even scientific paradigms. Peptides may be found that turn on expression of a specific gene, develop cell permeable peptides to avoid the need for gene therapy, or even make small-molecule screens using this principle. Of significance, in accordance with these approaches we attempt to bypass the need to understand the disease before effecting the treatment. In addition, theoretically, for some diseases a screen could be performed in a limited volume of a patient tissue to identify human-effective therapeutics for an otherwise incurable disease. E.g. a few mm³ piece of tissue (e.g. muscles for neuromuscular junction in the ALS), where a library was locally injected, could be followed by the biopsy of the library-containing tissue. One could then observe which peptides correlate with therapeutic effect (e.g. survival or rebuilding of neuromuscular junctions).

As a further example, consider nonalcoholic steatohepatitis (NASH), a common and complex disease partially characterized by fat accumulation in the liver. Since 1952, when Zelman reported liver inflammation and damage in patients with obesity, there has been significant advancement in understanding the mechanisms of NASH. Previous work found that the excess or impaired disposal of free fatty acids (FFAs) from the liver can result in the formation of lipotoxic metabolites and hepatocyte injury. Consequently, many drug candidates were developed under the hypothesis that reducing intrahepatic free fatty acids may ameliorate NASH. In particular, glucagon-like peptide 1 (GLP-1) agonists were intensively studied due to their ability to modulate insulin sensitivity, which relates to de novo lipogenesis and fatty acid β-oxidation in the liver. However, results on GLP-1 agonists for NASH have been largely inconsistent. To date, there are no FDA approved therapeutics for NASH, despite the availability of several putative targets such as GLP-1 receptors.

As such, a phenotypic driven paradigm in drug discovery has generated interest. This phenotypic approach to drug discovery fundamentally differs from target driven strategies by evaluating drugs based on their ability to modulate disease phenotypes rather than implicit molecular targets. Therefore, the phenotypic driven approach relies more heavily on the ability of a compound to demonstrate desirable biological properties first, and then to identify the molecular target that is responsible for these effects. This process effectively eliminates the bias of preconceived targets, which is especially important for complex diseases where there may be many molecular targets that have to be modulated in a specific manner to ameliorate disease. Additionally, while there have been advances in computational approaches for rational drug design, engineering molecules with complex polypharmocology remains challenging. Thus, in many cases, phenotypic based methods offer a more practical approach to engineer molecules with complex pharmacological profiles. Still, phenotypic driven drug discovery is largely serendipitous or relies on screening small molecules individually or in lower throughput compared to other screening methods.

To summarize these issues prior to discussing the present techniques, current target-driven approaches in drug development rely on understanding disease mechanisms before identifying therapeutics. This step requires significant effort, time, and resources. We would be able to identify new therapeutics first, without fully understanding disease mechanisms. Newly identified therapeutics may then provide insights into important aspects of pathophysiology. In this manner, we can rapidly develop effective therapeutics while still advancing our understanding of disease biology. However, many phenotypic-based strategies focus on screening small molecule libraries or have limited throughput. On the other hand, proteins and peptides can be used to generate highly diverse libraries, making them a suitable mechanism for agnostic screening. One technique for screening extracellular peptides, phage-display, may offer advantages for the future of mechanism agnostic drug development. However, phage-display assumes a molecular target is known and relies on affinity-based screening rather than linking sequence to biological phenotypes. To bridge this gap, we propose improving display screening methods and developing a platform for mechanism agnostic peptide screening (MAPS) based on disease phenotypes rather than binding affinity alone. For the purpose of explanation, we primarily focus on extracellular peptides, though it should be appreciated that other suitable molecular structures may be employed.

With this in mind, an alternative drug design pipeline is proposed that optimizes the prospective drugs for in vivo performance without a priori assumptions of the mechanism. Instead, we look for which drugs are most effective and safe in vivo from the start. This method is referred to herein as Mechanism Agnostic Peptide Screen (MAPS).

In particular, to overcome this initial challenge with target-driven drug discovery, we propose to develop a mechanism-agnostic screening platform to identify novel therapeutics without assuming prior knowledge of disease mechanisms. Specifically, we focus on adapting protein evolution strategies to engineer small peptide therapeutics. To achieve this, we will start by adapting display technologies to enable screening of extracellular peptides. Next, we will apply our peptide display approach along with computational methods to develop a mechanism agnostic peptide screening platform for identifying novel therapeutics in a model of nonalcoholic steatohepatitis. Through this work, we aim to accelerate the discovery of effective therapeutics while expanding our understanding of disease mechanisms of NASH.

To achieve this, genetically-encodable proteins and peptides are used as drugs. A gene delivery is performed into part of a diseased tissue. A different peptide or protein is then expressed in each cell. After a period of expression we biopsy the site of injection, and analyze the phenotype of cells for the presence of disease. In this context, such phenotypes can include, but are not limited to, visual appearance under a microscope, changes in gene expression, metabolism, or any other change identifiable at the single-cell level. The cells showing a positive resolution or mitigation of the disease are then analyzed to identify the DNA that led to a peptide/protein that mitigated the disease. The successful peptides/proteins are re-screened to confirm their performance as therapeutics. Once confirmed, the peptides/proteins are tested in a low-throughput context by delivering them to the tissue or cells of interest.

As discussed herein, in some cases the proteins/peptides are expressed intracellularly and the process screens for intracellular drugs. In some cases the proteins/peptides are displayed extracellularly on the cell surface during the screen. The extracellular part of the peptide, or a part thereof, is then used as an extracellular drug interfacing with the cell surface. In some cases these proteins/peptides are produced in cells through gene therapy. In other cases they are delivered through injection, oral, intradermal, inhalable, or other routes. In cases of intracellular proteins/peptides the delivery is intracellular. In the case of extracellular receptors, the delivery can be intracellular or to the tissue containing the cells of interest. In some cases the protein/peptides are used to screen for cell survival. For example, in cases of neurodegenerative diseases, or any other diseases resulting in cell loss, the cell survival after expression of the protein/peptide rather than phenotypic features of the cells can be used as a basis for considering the candidate drug successful. In some cases the cell phenotype will be evaluated using single-cell mRNA sequencing, in which case the mRNA encoding the protein/peptide will be used to identify the protein/peptide identity. In some cases the whole transcriptome will be used to evaluate which transcripts were changed by the cell, including the transcripts signifying cellular toxicity of the peptide. In some cases the recovery of cells containing expressed proteins/peptides can receive more than one copy of the DNA encoding the proteins/peptides. In some cases, different proteins/peptides will be encoded in the same cell. In that form MAPS allows for screening for protein/peptide drug combinations.

Further, in some cases we may aid the screening process with machine learning. While high-throughput screening allows for the fitness characterization of thousands of molecules, it may be laborious, time-consuming, and resource intensive. To address this, in silico directed evolution may be employed to optimize the fitness of experimentally characterized molecules. I propose to use a “low-N” directed evolution paradigm, where a machine-learning model is built hierarchically starting from global information of all functional proteins and peptides, then tuned with evolutionarily or significantly similar peptides to those that are experimentally characterized, and finally trained on mechanism agnostic peptide screening data.

In MAPS, we will test many randomized peptides to identify those with unknown functions. In some cases, this will result in a collection of characterized peptides with low sequence similarity, making sequence alignment and other analyses challenging. I propose to use a vector-clustered multiple sequence alignment combined with the “low-N” approach in these cases for optimized sequence homology searches. This would allow in silico directed evolution of a target class of peptides by only characterizing a small number of peptide candidates by experimental screening, further accelerating drug development. Advantages of the presently disclosed techniques include, but are not limited to: lower cost by bypassing in vitro testing and streamlining lead optimization.

Possible applications of the presently described techniques include, but are not limited to, identification of therapeutics for diseases are characterized by cellular phenotype, such as neurodegenerative disorder (mitigating cell loss), metabolic disease, infectious disease (preventing cell entry), resilience to toxins or environmental factors. In addition, possible applications may include controlling any biological process that is dependent on cellular phenotype, such as immune cell activation, brown fat thermogenesis, cell proliferation.

With the preceding in mind, the technology described herein relates to a mechanism-agnostic platform for drug discovery that relies on the observation of phenotypic changes to identify potential therapeutics. By relying on a phenotype-driven method, the potential for therapeutic candidates to have poor activity is eliminated. Aspects of the proposed platform are described below.

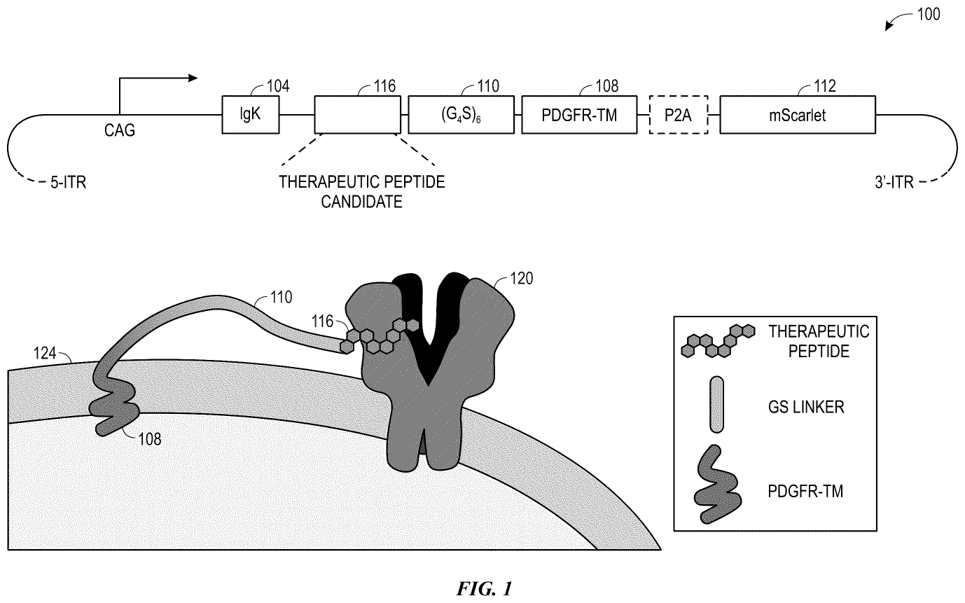

In accordance with certain aspects of the present approach, engineered extracellular protein carriers (EPC) are employed. As used herein EPCs are genetically-encodable, cell-surface tethered systems that allow for the restriction of peptide therapeutics 116 to a cell surface 124, where they can interact with extracellular receptors 120. In one embodiment an EPC 100 comprises an Ig-κ secretion leader sequence 104, platelet-derived growth factor receptor β (PDGFR-β) 108, a flexible linker 110, and a fluorescent protein 112. An example of one such EPC structure 100 is illustrated in FIG. 1.

As illustrated in FIG. 1, upon interaction of the peptide candidate 116 with an extracellular receptor 120, phenotypic changes that are associated with potential therapeutic benefits may be observed, which will allow for additional study. To maximize the efficiency of this system, multiple protein candidates 116 may be screened simultaneously to expedite drug discovery. Each peptide candidate 116 may be expressed in a single cell, but cells with the expression of multiple peptides may allow for the discovery of combination therapies. Cells with notable phenotypic changes will be separated out and processed for RNA extraction, as well as library preparation for sequencing. This will allow for the identification of the peptide sequences, which can be further studied through in vitro and in vivo studies.

In a further aspect, a HepG2 cell sorting method may be employed. This portion of the screening method uses Percoll gradient centrifugation and fluorescent dyes to separate out cells with differing levels of intracellular lipid accumulation based on their densities. In particular, such a screening pipeline may be employed for a non-alcoholic steatohepatitis (NASH) model. The use of Percoll gradient centrifugation and fluorescent dyes in this context has been confirmed as a way to separate HepG2 cells based on the phenotype of interest (i.e., level of intracellular lipid accumulation).

With the preceding in mind, presently conducted research related to the present techniques may be described as follows.

Develop functional genetics screening platform to engineer novel extracellular peptide therapeutic candidates for steatohepatitis—With respect to this aspect, focus is on developing a platform to screen peptides with extracellular modes of action based on disease phenotypes. To achieve this, extracellular peptide carriers (EPCs) are engineered for genetically encodable cell-surface tethered expression of peptide therapeutics, as shown in FIG. 1. By restricting therapeutic peptides to the cell surface, peptides can bind to extracellular receptors, while still attached to their parent cell. Thus, combining EPCs with current single-cell screening methods such as fluorescence-activated cell sorting allows for high-throughput assays of therapeutically relevant peptides that bind extracellular receptors. The milestones for this aim will be 1) engineering EPCs and validating the efficacy of cell-surface restricted peptides as therapeutics and 2) applying EPCs for screening therapeutic peptide candidates with lipid lowering effects in liver hepatocytes.

Engineer extracellular peptide carriers for genetically encodable, cell-surface tethered expression of peptide therapeutics—While there have been advances in gene therapies, several challenges regarding gene delivery in humans, including invasiveness and high cost, limit their accessibility which is not ideal for common diseases like NASH. Therefore, therapeutics that avoid the need for complex gene delivery, such as peptides with extracellular modes of action, are attractive. Here, we aim to engineer an extracellular peptide carrier (EPC) to restrict peptides by tethering to the cell membrane using flexible linkers. Similar technologies like phage display and drugs acutely restricted by tethering (DART) have been developed. Unlike phage display, peptides are tethered to the cell surface by a long flexible linker, allowing for cell surface receptor binding while still restricted to their parent cell. Unlike DART, focus is specifically on tethering peptides and proteins as opposed to small molecules. As such, EPCs are fully genetically encodable, enabling cell type and temporal specificity.

EPC vectors (pAAV.EPC and pLenti.EPC) were constructed based on a modified pDisplay expression vector, using an Ig-κ secretion leader sequence and platelet-derived growth factor receptor β (PDGFR-β) transmembrane anchor. To allow for peptides to bind to extracellular targets, a long flexible linker was included to tether peptides to a transmembrane anchor. Our initial EPC vectors use a GGGGS (i.e., G4S) motif in the linker with a length based on a minimum distance calculation. However, to empirically determine the optimal linker length, we constructed several versions with glycine-serine linkers ranging from 1.75 to 12.25 nm.

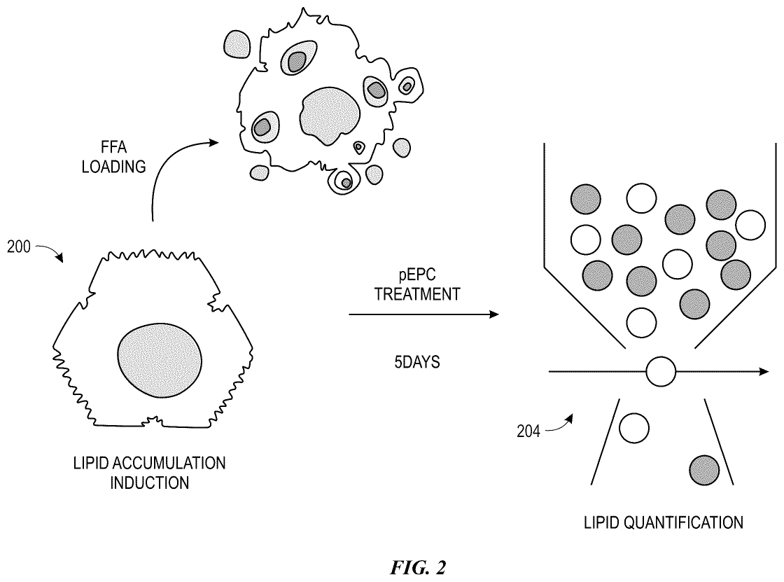

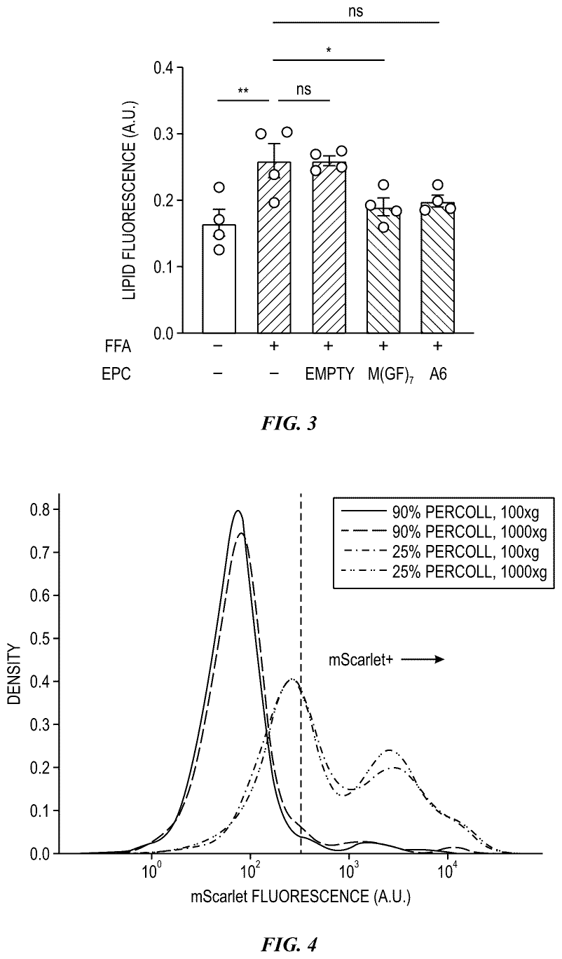

To test the functionality of cell-surface tethered peptides, a simple in vitro model of steatohepatitis was used, as depicted in FIG. 2. In this model, HepG2 cells 200 are made steatotic by loading the growth media with FFAs (500 μM 1:1 oleic: palmitic acid) for 3 days. Lipid accumulation in HepG2 cells can then be quantified (step 204) using lipid specific fluorescent dyes and epi-fluorescence imaging or flow cytometry. We performed transient transfection of pEPCs containing sequences for the 18 amino acid AWRK6 peptide, which is known to reduce lipid accumulation in liver hepatocytes. Additionally, we tested poly-glycine peptides consistent with previous reports suggesting glycine biosynthesis impairment in NASH and the glycine tripeptides which has been investigated for NASH. We observed a statistically significant decrease in lipid fluorescence in HepG2 cells expressing tethered peptides compared to empty EPC vectors or an mScarlet reporter, as shown graphically in FIG. 3. While positive control peptides showed efficacy in reducing lipid accumulation, it's possible that our initial linker length is not optimal for this particular system (30 a.a.≈10.5 nm). In cases where linkers are too short to allow for peptide binding to extracellular receptors, we expect to observe smaller changes in cell phenotypes. As the linker length increases and approaches an optimal length, we expect to observe larger changes in cell phenotypes. Beyond an optimal linker length, the local concentration of peptides near their parent cell may decrease and increased off target effects may be observed in the case where peptides from one cell bind to neighboring extracellular receptors. To test our hypothesis, we will quantify lipid fluorescence in steatotic HepG2 cells expressing positive control peptides with different linkers ranging from lengths of 1.75 to 12.25 nm.

Alternative Strategies—While cell-surface expression of therapeutic candidates showed statistically significant reduction of lipid accumulation, we observed low transfection efficiency of plasmid DNA of ˜15-20% in these experiments. This is consistent with other investigations using HepG2 cells. To further investigate the efficacy of EPCs, these experiments will be repeated with EPC mRNA transfection, which has been reported to have higher efficiency for HepG2 cells.

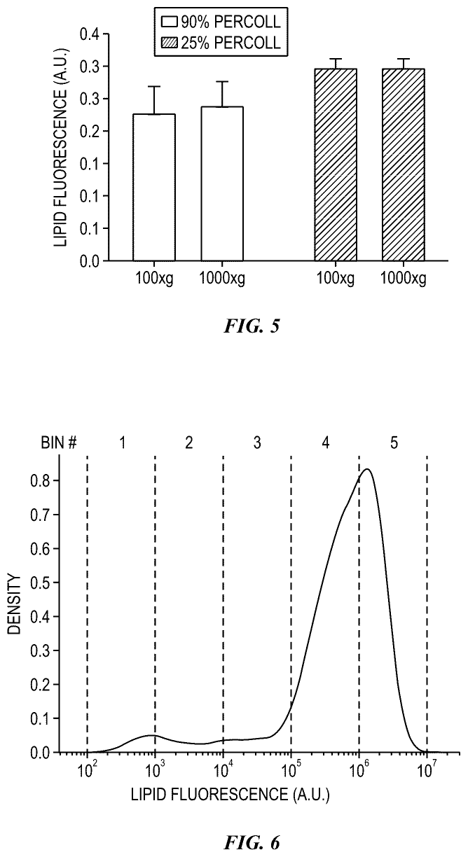

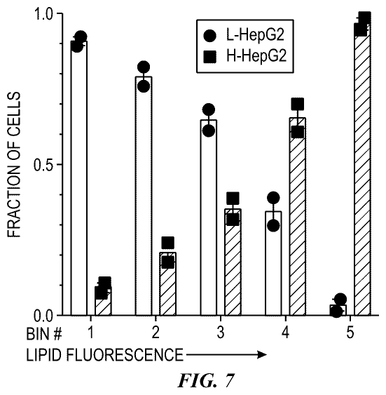

Develop a screening pipeline for in vitro NASH model—Extracellular peptide carriers as described herein enable screening of functional peptides with extracellular modes of action by restricting them to individual cells. In this aspect, developing a screening pipeline for engineering peptide therapeutics for steatohepatitis is described. Here, the main phenotype targeted for steatohepatitis is intracellular lipid accumulation. Previously, cell-surface peptide tethering using the 18 amino acid AWRK6 and 8 amino acid poly-glycine peptides was tested, which reduces lipid accumulation in liver hepatocytes. To better understand the sensitivity of screening HepG2 cells based on lipid accumulation, an initial round of sorting was performed to determine how well we can separate steatotic vs. wild-type HepG2 cells. Hepatocyte density and size increases with increased levels of intracellular lipids. Thus, cells may be separated based on lipid accumulation by using lipid specific fluorescent dyes and differences in cell densities. To test this approach for hepatocyte screening based on intracellular lipids, two populations of HepG2 cells were generated: (1) a “low-lipid” HepG2 population (L-HepG2) which consisted of wild-type cells grown in normal culture media, and (2) a “high-lipid” HepG2 population (H-HepG2) consisting of cells made steatotic as previously described. A 1:1 mixture of L to H-HepG2 was made by sorting equal numbers of cells by fluorescence activated cell sorting. Mixed HepG2 cell populations were re-separated in bulk based on density by percoll gradient centrifugation. Each fraction of cells collected after percoll centrifugation were stained with a fluorescent lipid dye and analyzed by flow cytometry, the results of which are illustrated graphically in FIGS. 4 and 5. For each sample, 5 equally log-spaced bins along the lipid distribution were analyzed, as shown in FIG. 6. For cells processed by a 90% percoll gradient, we observed majority L-HepG2 cells in the lowest bin corresponding to cells with the lowest intracellular lipids, and about 2% H-HepG2 cells as error, as shown graphically in FIG. 7. Similarly for the bin with the highest lipid fluorescence, we observed recovery of 98% H-HepG2 cells, suggesting lipid fluorescence and bulk separation by percoll gradient centrifugation may enable screening of steatotic liver hepatocytes.

In practice, HepG2 cells expressing peptide candidates with lower fitness may not fully reduce intracellular lipids to the level of wild-type cells. Therefore, the ability to screen HepG2 cells with additional varying levels of lipid accumulation may be tested. In such a test, populations of HepG2 cells at a range of FFA concentrations from 0 to 1 mM in steps of 100 μM (i.e., 0, 100, 200, . . . , 1000 μM) will be generated individually. Each sample will be labeled by a fluorescent reporter with an 8-nucleotide barcode corresponding to the amount of FFA used to induce lipid accumulation. For each labeled group, a mixture will be generated by sorting a prespecified number of cells to better control the expected number of cells in each condition. The mixed population will be processed by percoll gradient centrifugation and FACS to collect cells from 5 equally log spaced bins along the lipid distribution. RNA extraction will be performed followed by cDNA synthesis and PCR amplification of fragments containing sample specific barcodes. Amplified fragments will be sent for NGS and analyzed to estimate the number of cells from each FFA group per bin.

In vitro screening of therapeutic peptide candidates for steatohepatitis—Lowering of glucose and lipids by glycine and leucine have been reported. By way of example, it was found that DT-109, a glycine-leucine tripeptide, lowers glucose and lipids more effectively than glycine alone. Additionally, a reduction in intracellular lipid concentration in vitro was observed after administration of 8 a.a. poly-glycine peptides. It is therefore hypothesized that there may be peptides with combinations of leucine or glycine with other amino acids that also reduce intracellular lipids. To screen for alternative peptides that incorporate different amino acids in an 8 a.a. poly-glycine peptide, we will perform randomized in silico mutagenesis and sample 1000 variants to synthesize in an oligonucleotide pool for cloning into our EPC lentivirus vector backbone with an inducible TRE promoter. After sequencing confirmation, the library will be packaged in a lentiviral vector (LV) for efficient gene delivery. For screening, the lentivirus library will be administered at a multiplicity of infection (MOI) of 0.3 to ensure single peptide expression. More generally, the MOI may be between about 0.01 to about 100. Additionally, the LV library will encode for bsd to allow for blasticidin selection of transduced cells. This ensures that most cells will express a peptide from the delivered library. Lipid accumulation will be induced in selected cells as previously described and peptide expression will be induced by doxycycline. Cells with the lowest and highest 5% lipid fluorescence will be sorted and processed for NGS to recover peptide sequences. Recovered peptides will be tested in low throughput individually to validate their activity. Functional peptides will then be diversified and used for a subsequent round of screening.

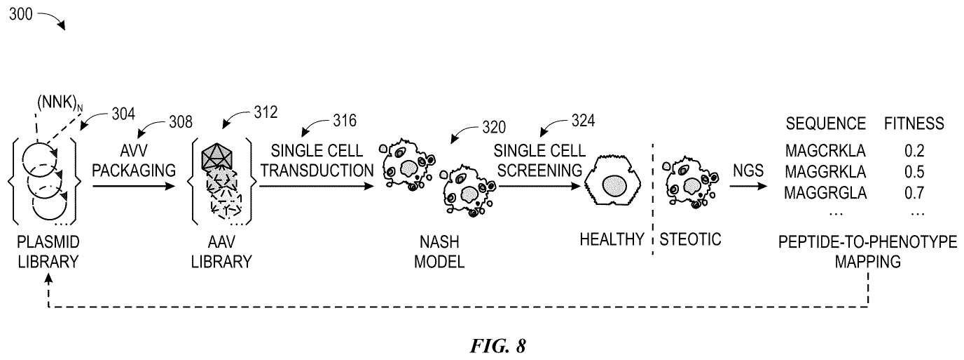

High-throughput screening of diverse peptide libraries for mechanism agnostic therapeutic discovery—The first rate limiting step in most drug discovery campaigns, is mechanism discovery. To avoid needing specific disease targets for therapeutic discovery, we hypothesize that diverse peptide libraries are likely to contain variants that are effective for a wide range of druggable targets and thus, diseases. Additionally, for diseases with multiple druggable targets, it is still difficult to design drugs with complex polypharmacological signatures despite advancements in computational drug design. Consequently, screening fully or partially randomized peptide libraries may provide an alternative strategy for identifying molecules with complex pharmacological signatures that otherwise would be difficult to design upfront. In this aspect, the screening pipeline 300 described herein will be applied to libraries of randomized peptides which are not based on a specific parent peptide or molecular target, as shown schematically in FIG. 8. As shown in FIG. 8, an overview of mechanism agnostic peptide screening is schematically illustrated. In this example, diverse DNA libraries 304 encoding randomized peptides are packaged (step 308) in viral vectors for efficient gene delivery. Viral vector libraries 312 are applied (step 316) to a NASH disease model 320 at a low multiplicity of infection for single cell tethered peptide expression using EPCs. Cells are screened (step 324) based on lipid accumulation and sequenced to recover peptide information. In this manner, we aim to identify novel peptides without assuming specific mechanisms for NASH, including impairment of glycine biosynthesis or insulin sensitivity. In this manner, the present techniques may be shown to be effective in contexts where little is known about disease mechanisms. A milestone for this aspect may be the identification of ≥1 peptide with lipid lowering properties in vitro.

In vitro screening of diverse peptide libraries—To identify potentially novel peptides without assuming glycine biosynthesis impairment or other mechanisms play a role in NASH, we propose to screen a randomized 8 a.a. peptide library. This is based on work in adeno-associated virus (AAV) capsid and MHC class I peptide screening which generally target 7-9 residues. Additionally, lengths of most currently FDA approved synthetic peptides are generally 2-10 a.a. However, beyond 9 a.a., the theoretical degeneracy of a fully random library is greater than 20⁹, which adds additional technical challenges. Randomized 24-bp libraries were constructed by PCR using an oligonucleotide primer with degenerate bases (NNK). PCR products were inserted into pAAV.EPC and pLenti.EPC by Gibson Assembly. Library insertion was validated in bulk by sanger sequencing and in the case of AAV vectors, AAV invertible terminal repeats were checked for integrity by SmaI digests. Each library will be packaged in either AAV9 or lentivirus using the generation 3 lentiviral packaging system. Libraries will be administered to HepG2 cells at a low MOI (0.3) to ensure single peptide expression from each transduced cell. Afterwards, we will perform blasticidin selection of transduced cells and induce lipid accumulation as described herein. After lipid accumulation, we will induce gene expression of EPCs containing random 8mer peptide sequences and screen cells by FACS to recover peptides expressed in cells with the lowest and highest 5% lipid fluorescence. Recovered cells will be processed for RNA extraction and library preparation for sequencing. The top peptide sequences will be validated in low throughput in vitro. Functional peptides will then be diversified further and used for subsequent rounds of screening.

In vitro and in vivo validation of top therapeutic peptide candidates—Extracellular peptide carriers enable screening extracellular peptides by restricting them to individual cells. However, for practical use of these peptide drugs, it would be beneficial to identify those that do not require the additional tethering by EPCs. Once the top peptides are selected based on the studies described here, we will validate their effectiveness by direct administration. Each peptide candidate will be synthesized and confirmed by mass spectrometry. These peptides will then be administered in the growth media for steatotic HepG2 cells at 0.01 to 0.1 μM. Peptides will be evaluated based on quantification of lipid accumulation as well as triglycerides, low density lipoprotein (LDL), high density lipoprotein (HDL) and total cholesterol. Additionally, while in vitro validation is useful, the mechanisms of NASH are complex and may not be fully realized in simple in vitro models. Thus, we will use rodent NASH models to better characterize the generalizability and efficacy of peptide candidates identified as discussed herein. While rodent models also do not fully recapitulate human NASH, they provide an alternative model for testing peptides that may work through conserved mechanisms. Additionally, there are many rodent models for NASH that each focus on different aspects of human NASH and can be easily developed through dietary or genetic manipulation. For this analysis, we will focus on a dietary NASH model which induces lipid accumulation and further hepatocyte ballooning and fibrosis. To achieve this, C57BL6/J mice will be fed a choline-deficient L-amino acid-defined, high fat diet consisting of 60% kcal % fat and 01% methionine by weight, ad libitum for 12 weeks. After disease induction, peptide drugs will be delivered by intraperitoneal (i.p.) injection daily for an additional 12 weeks. Livers will be collected for quantifying lipid accumulation, triglycerides, low density lipoprotein (LDL), high density lipoprotein (HDL), total cholesterol, alanine transaminase (ALT), and aspartate transaminase (AST).

Alternative strategies—Drug validation with a 12-week disease induction followed by daily i.p. peptide administration is proposed for consistency with prior studies. However, it is possible that depending on peptide composition, other routes of administration are better suited due to either half-life or other properties. If needed, an alternative delivery method may be employed, such as intravenous administration of peptides. Further, if peptides are not effective by direct injection, we will repeat experiments by delivering viral vectors harboring sequences for tethered peptides. This will help identify potential optimization steps that are needed to increase binding affinity in cases where peptides are not effective at lower local concentrations or other possible challenges with direct peptide administration.

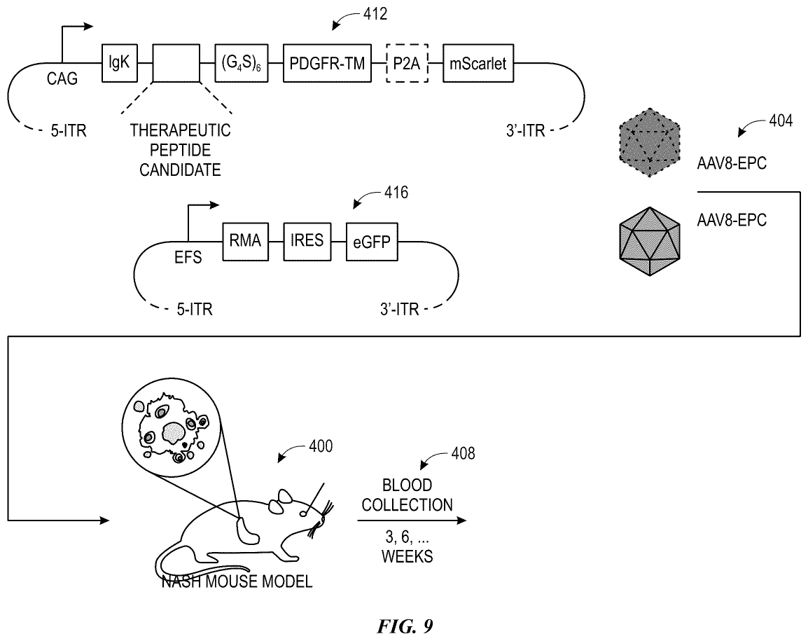

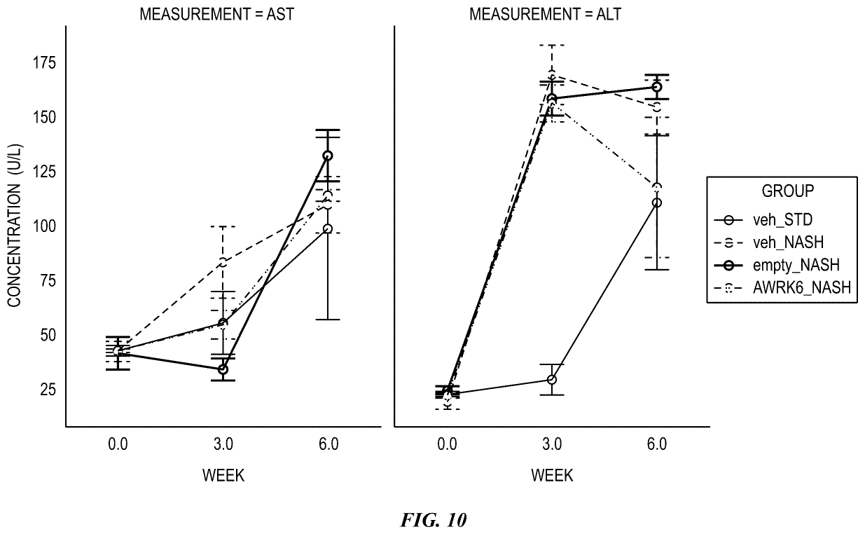

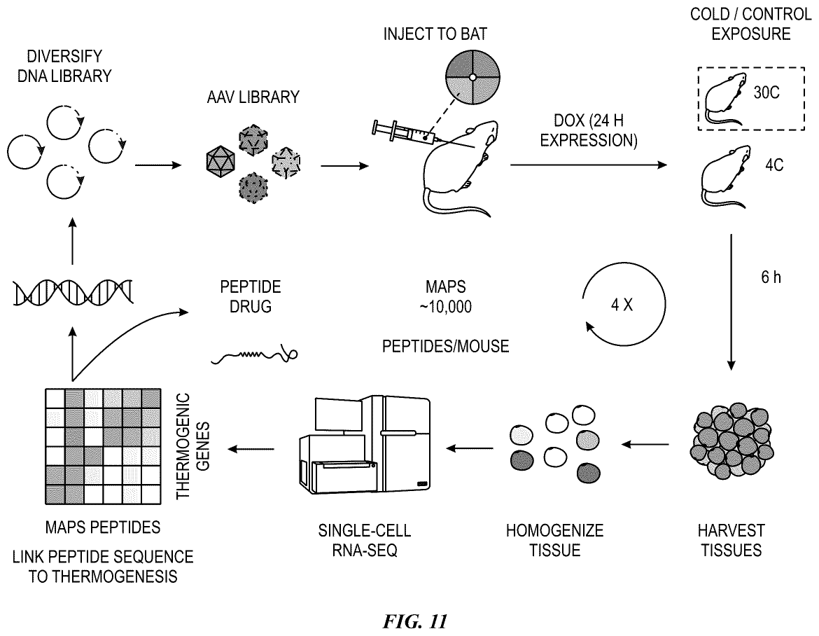

Overview of in vivo validation of extracellular peptide carriers (EPCs)—Turning to FIG. 9, C57BL6/J mice 400 were placed on either a standard diet or 60% kcal fat, 0.1% methionine, choline deficient NASH diet (n=7 or 8). Mice 400 were injected with AAVs (step 404) containing sequences for an 18 amino acid therapeutic protein AWRK6 (412) or an empty control (416) containing only the transmembrane anchors. Blood was sampled (step 408) two weeks after AAV delivery (week 0) and then every 3 weeks. Turning to FIG. 10, aspartate transaminase (AST) and alanine transaminase (ALT) were measured from serum as shown in the depicted graphs. At 3 weeks after NASH diet induction, ALT levels are elevated for all groups on NASH diet. At the 6-week timepoint, ALT levels increase for the vehicle group on standard diet. However, ALT decreases to comparable levels for NASH mice treated with AAVs harboring the therapeutic protein, AAV8-EPC-AWRK6.

Single-cell transcriptomics of therapeutic peptide candidates—The effects of peptide drugs in NASH mice at the transcriptome level may be further investigated. By identifying specific markers that are modulated in response to peptide administration, we can rationalize potential new mechanisms of action or confirm existing ones. The same workflow as described herein with respect to In vitro and in vivo validation of top therapeutic peptide candidates may be repeated for in vivo validation of peptide drugs and for recovering livers of NASH mice treated with or without the top peptide candidates. The livers will be homogenized and prepared for library construction and single-cell RNA sequencing. For analysis, we will quantify the top differentially expressed genes between NASH, wild-type, and treatment groups to identify potential targets for each peptide.

Alternative strategies—Single-cell RNA sequencing provides a highly detailed view of the transcriptome; however, it may not allow us to observe small changes due to the large amount of noise that can arise from sequencing. If challenges with this method are encountered, we will use bulk RNA-sequencing or proteomic approaches to investigate differences at the protein level.

Develop peptide fitness prediction models for exploring vast sequence space—The theoretical diversity of an NNK library targeting 8 residues is over 25 billion sequences. This library will span most of the sequence space for an 8 amino acid peptide. However, in practice less than 1% of the library may be sampled due to bias in construction steps, viral vector packaging limits, and experimental conditions like cell culture size. Although works on viral vector engineering routinely use NNK randomization of up to 7 amino acids in capsid proteins, screening transduction events through fluorescence or Cre based methods may be easier than subtle changes in disease phenotypes. Thus, much of our library may be unexplored and only a small fraction of peptides that are functional may be recovered. Machine learning models trained on assay labeled data of peptide candidates for NASH treatment and in silico screening may be employed to potentially identify novel peptide therapeutic candidates missed during earlier screening. The milestone for this analysis may be: 1) test accuracy ≥80% on a classification task using assay labeled data, and 2) identification of ≥1 peptide candidate with mean predicted fitness greater than the most fit peptides found experimentally.

Train convolutional neural networks on labeled randomized peptide datasets—Machine-learning directed evolution (MLDE) is suitable for integration with MAPS. While there are many approaches for protein fitness prediction that rely on a hybrid of unsupervised training based on evolutionary properties and supervised fine-tuning, supervised fitness prediction may be initially evaluated. For screening randomized peptides, it is possible that some recovered sequences will have low similarity to known peptides, therefore limiting the utility of evolutionary based methods that use either local or global sequence information. Convolutional neural networks (CNNs) are effective at mapping sequence to fitness. To enable additional virtual screening of peptide candidates, we will train 1D CNNs on datasets as discussed herein with respect to in vitro screening of diverse peptide libraries and in vitro screening of therapeutic peptide candidates for steatohepatitis and estimate accuracy by 5-fold cross validation. Rather than training CNNs on a regression task using count based log-enrichment of sequences from both high and low lipid containing HepG2 cells, we will employ model based enrichment estimation (MBE) instead. For our particular task, the standard count based log enrichment may be represented as:

$$log {e}_{i} = \frac{{n}_{i}^{B} / {N}^{B}}{{n}_{i}^{A} / {N}^{A}} \qquad ( 1 )$$

where nᵢ^{A} and ng are read counts for the i^{th} sequence in libraries A and B. The total size of the libraries NA and NB are used for normalizing read counts. In our screening experiments, nᵢ^{A} may be sequences from cells with the lowest lipid accumulation below a given threshold, and nᵢ^{B} are sequences from cells with the highest lipid accumulation. On possible complication with this type of count based log-enrichment is high variance in cases where nᵢ is low, which is usually the case with negative and counter screens. To account for this, we propose to instead solve a density ratio estimation problem by MBE to estimate log enrichment. This approach will be applied with 1D CNNs and we will test the effects of activation functions, kernel size, and layer depth, to identify optimal architectures for fitness prediction given our specific datasets. Additionally, encoding has been shown to affect mean fitness achieved in MLDE simulations. We will initially start with Georgiev encodings, as it has been shown to improve mean fitness compared to simple one-hot encoding.

Alternative strategies—Generally, CNNs and other neural network architectures benefit from large training sets. It is possible that most peptide sequences screened in the previous described analyses will have poor fitness. In most cases, training networks on sequences with zero fitness allows for the model to learn how to predict nonfunctional sequences, which is not desirable. Therefore, if we are limited by training data and accuracy is below 70%, we may employ a hybrid approach of using an evolutionary model or larger language model such as ESM2. As noted previously, the major limitation with these is high quality MSAs will likely be difficult to construct with most alignment algorithms. If we recover sequences that have low similarity to known peptides used for pretraining, we will construct MSAs using the vector clustering MSA algorithm (vcMSA). vcMSA was developed as an alternative alignment algorithm and has been shown to improve alignment between low-similarity sequences.

In silico evolution of diverse 8mer peptides—Machine learning guided evolution enables rapid screening of protein variants. We will perform 2000 simulations of evolution by applying our CNN regressor on sequences from an (NNK) & distribution. For simulation, we will test two sampling approaches. First, we will start by randomly sampling 200,000 sequences from the (NNK); library, excluding those that have already been recovered during screening. As a second approach to avoid sampling in non-informative holes, we will generate sequences from a generative adversarial network (GAN) based on ProteinGAN. To generate sequences, we will perform one round of zero-shot learning on 200,000 randomly selected sequences using a CNN classifier as described herein. The top sequences will be used as parents for ProteinGAN to generate the next round of parent sequences. Generated sequences will be diversified to explore local sequence space in the subsequent round of in silico evolution. For each round, peptides will be ranked by fitness scores and the top 0.001% will be selected and diversified to generate secondary libraries. In silico screening will be repeated until the max fitness level stops increasing. We will calculate the mean fitness and recover the top 100 peptide sequences with the best fitness scores across all simulations. We will experimentally validate the sequences with predicted log enrichment scores greater than the top peptides recovered from screening using the same HepG2 NASH model as previous aims.

As discussed herein, the proposed work combines experimental and computational protein engineering strategies to expand on approaches for screening extracellular peptides as therapeutics in high throughput. We mainly focus on NASH as a model disease due to its potential impact and current progress in developing effective therapeutics with known mechanisms to validate our findings. If successful, however, this platform may be used for any disease with observable single-cell phenotypes. It may be especially useful in cases where diseases mechanisms are unclear. Additionally, MAPS can be easily adapted for screening intracellular peptides as well, for example in cases where intracellular peptide-protein interactions are required.

While the preceding describes the presently contemplated MAPS process both in general and in the context identifying and developing therapeutics for NASH, it should be appreciated that other applications are contemplated. For example, improving service members' performance in cold climates requires a rapid and effective thermal adaptation. Brown Adipose Tissue (BAT) adapts to cold over several days of training to generate heat upon cold exposure, but even that adaptation cannot prevent many of the cold's adverse effects, e.g. loss of dexterity, concentration, or muscle function.

To enable improved arctic resilience we will use a new directly-in-tissue high-throughput screening method, as discussed herein, to identify drugs that induce a better reversible BAT thermogenesis that satisfy 5 design constraints: 1) enable rapid, reversible, adaptation to cold (<5 C) within 24 h; 2) Increase the intensity of BAT thermogenesis beyond humans' basic capability, 3) are systemically administered e.g. with a hypodermic needle, 4) are non-genetic, cheap, and scalable with available production and distribution chains. 5) can be rapidly (˜3 y) translated to large animal studies. Current efforts enable cold adaptation through lengthy training. Alternatively, known cold-adaptation pathways can be activated through growth factors and hormones, however they typically: 1) require discovery of underlying mechanisms which delays the drug development, 2) have adverse effects by acting on off-target tissues, 3) lack scalability for widespread production and storage, 4) activate natural pathways that adapt BAT to the extent intended for humans—not beyond it.

To achieve our 5 goals, we will discover better thermo-adaptive drugs by screening for thermogenic effects directly in vivo in BAT and evolve injectable non-genetic drugs to rapidly induce or shut down the cold adaptation. As discussed herein, this process of screening is referred to as Mechanism Agnostic Peptide Screening (MAPS). In MAPS, every cell is a single experiment, which means we can test large numbers (>10,000) of drugs in a single animal. We use peptides as drugs, which means they can be optimized by directed evolution and screening in vivo. The peptides are extracellular, so they can be administered systemically (see e.g. insulin). Because MAPS is performed directly in live animals and tissue of interest it optimizes in vivo performance, acts directly upon the BAT reducing off-target effects, and accounts for variables that would be missed in vitro. Once MAPS is done, top 5 candidates will be tested in mice to measure behavioral and physiological cold adaptation and its reversal. In parallel, an in vitro screen will be done to de-risk in vivo studies.

By way of further context, the arctic may soon become a strategically important source of resources and a major shipping route due to the climate change. The ability of the US military to rapidly respond to a crisis or conflict will rely on the preparation of the personnel to the arctic environment and the mainland. Typically, cold adaptation is a slow process of unpleasant physical training that often takes days to weeks. Even if successful, the effects of such adaptation are limited. For example, BAT-mediated non-shivering thermogenesis (NST) produces on average only ˜12% (range 0-30%) of the basal metabolic rate. The more effective skeletal muscle-mediated shivering thermogenesis causes uncontrolled muscle movements which affect dexterity and focus. Unfortunately, the cold adaptation alone does not improve dexterity in the cold, possibly due to its modest ˜36% reduction in shivering. Ideal cold-adaptive drugs would improve adaptation beyond what is naturally achievable for humans.

Cellular basis of cold adaptation and available therapeutic strategies—Cold adaptation results in many cellular and molecular changes in the body, including increase in the BAT's volume and heat generation. The generation of heat in BAT occurs through NST and is carried out mainly by the brown adipocytes. The total extent of heat production depends on the number of brown adipocytes, their metabolic activity, and their vascularization. Other cell types, including beige adipocytes and white-adipose tissue (WAT), can also produce heat through NST after prolonged cold adaptation. NST in these cells is activated through multiple steps, including breaking down and recruiting of fatty acids, which serve as substrates for the uncoupling protein 1 (Ucp1) to generate heat in the mitochondria. NST is activated through endocrine and peripheral nervous systems, including through molecular factors. While these factors can activate BAT-mediated NST, they may take a few days to achieve designable effects and lead to significant and sometimes dangerous side effects, likely because they are not exclusively affecting BAT. Consequently, as of yet, there is no ideal therapeutic target that can safely and effectively increase the NST.

Improving drug discovery to develop thermo-adaptive therapeutics—To improve the cold adaptation and NST generation in the cold, the current drug development paradigm may need to be improved, as discussed elsewhere herein. Current approaches have a number of problems that lead to low rate of translation and a lengthy and rapidly rising cost of drug development. Some of the problems include: 1) many identified drugs work well in vitro, but not in animals or humans, yet the majority of initial drug screens are done in vitro; 2) basic biology knowledge is needed to even start the drug development; 3) in vitro, small animal, large animal models need to be available; 4) The drugs are designed to target a single receptor, but pathogenesis is often complex, involving multiple receptors, as is the case in cold adaptation. Therefore, as discussed herein, a new drug development paradigm is needed to alleviate these problems and find more effective cold-adaptive therapeutics. As discussed herein, this may involve Mechanism-Agnostic Peptide Screening (MAPS) an example flow of which is schematically illustrated in FIG. 11. As discussed herein, this proposed process will: 1) substantially speed up the process of identifying drugs 2) increase the chance of identifying drugs that are functional in vivo, 3) enable drug discovery even if the underlying biology knowledge is limited, 4) work in both large and small animals. 5) allow for additional high-throughput optimization of desirable characteristics such as tissue-specificity.

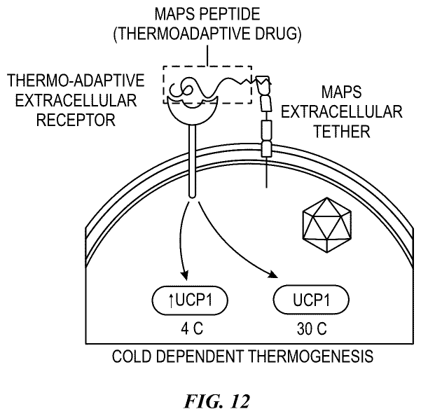

With reference to FIG. 11, the principles and design of one implementation of Mechanism Agnostic Therapeutic Screening (MAPS) is schematically illustrated. In this example, a library of random peptides is expressed on the surface of BAT adipocytes. Mice are then challenged with cold; After 6 h their tissues are harvested and homogenized into single cells. Single-cell sequencing is used to measure thermogenic gene activity and link it to the RNA encoding the peptide that induced the thermogenesis. The top ˜10 most thermogenic peptides are further mutated and subjected to 3 more rounds of evolution. Further aspects are illustrated in FIG. 12, which illustrates that MAP peptides are evolved to act through extracellular interaction with receptors on BAT adipocytes. (The peptides are tethered to a transmembrane domain on a flexible linker to avoid steric hindrance). After screening, the extracellular portion becomes a systemically-injected drug optimized to act on BAT cells' surface. In such example, peptides are injected subcutaneously (SQ) and animals' cold adaptation (e.g., core temperature, dexterity, coordination, and/or cold avoidance) is tested in vivo. Once cold-adaptive drugs are identified, they are injected into mice to induce cold adaptation, and MAPS screen is performed again to find drugs that can reverse it.

As discussed herein, a present goal is to develop systemically non-genetic, injectable, peptide-based drugs that can induce stable and reversible cold adaptation faster and more effectively than the training protocols. MAPS is a platform for drug development that can be applied to many disorders and endpoints, as long as they change a cell-level phenotype. It is compatible with both large and small animals. MAPS, in some embodiments, uses peptides as drugs because their production and storage are well-established. If successful, MAPS could be used, e.g. to prevent or treat neuronal degeneration (due to e.g. blast injury), render cells immune to infections, or prevent demyelination.

With the preceding in mind, one goal is to develop systemically injectable, non-genetic, scalable therapeutics that induce a stable adaptation of BAT to and from cold environments within 24 hours. To achieve this, we propose a new therapeutic development pipeline, called MAPS, as discussed above and shown in FIG. 11. In MAPS every cell in the tissue is an independent experiment, and consequently tens of thousands of peptide drugs can be tested in a single mouse. MAPS is performed by gene delivery to express randomized peptide library in BAT—a different peptide in every cell. The randomized peptides-drug candidates—are then displayed on the outside of the BAT cells, as shown in FIG. 12. Some of these peptides then interface with the receptors on the cell surface and modulate cold adaptation. Expression of peptides is induced for only 24 hours, after which mice are exposed to cold (4 C, 6 h). Immediately afterwards, their BAT tissues are extracted, BAT adipocytes isolated, and subjected to single-cell RNA sequencing. Using the sequencing, we measure activity of thermogenic genes in each cell, and associate it with the mRNA sequence encoding the MAPS peptide. We then select the peptides that led to most efficient thermogenesis compared to controls. These best peptides are then mutated further, and the process of MAPS is repeated ˜3× to optimize the peptides' thermogenic properties. If needed, to further improve the safety and avoid side effects e.g. hyperthermia, MAPS can also be used to test the top thermogenic peptides in high-throughput to find drugs with reduced peripheral effects, increased speed of action, stability, or only inducing thermogenesis upon cold exposure.

The extracellular portion of the 5 (or other suitable number) most cold-adaptive peptides will be chemically synthesized and injected into mice to evaluate their effects on cold adaptation using measures relevant to the service members. Afterwards, an additional screen may focus on finding drugs reversing the cold adaptation. The project contains three tasks: 1) in vitro screen for de-risking in vivo studies, 2) in vivo screen, 3) in vivo validation.

In vitro screening for thermoadaptive therapeutics—MAPS uses high-throughput screening and directed evolution to develop new drugs in vivo. This principle has been applied to a plethora of proteins, but does not appear to have not been used in vivo for drug development. Consequently, as a de-risking strategy, we may perform in vitro and in vivo screens in parallel.

Construction and expression of the library—As a starting point we will use a positive control protein with limited cold-adaptive properties—IL6. We will mutate IL6, one mutation per clone, and screen for improved cold adaptation. In parallel, we will also synthesize a library of completely randomized peptides. We will use 8-amino acid peptide length. That random library could target receptors that induce rapid cold adaptation but are not yet known. Here, the peptides will be displayed on cell surface to screen for extracellular mode of action. For therapy, the extracellular portion will be synthesized and injected systemically.

High-throughput screening in vitro—In one study we will perform the screening with approximately a thousand randomly selected clones per round of evolution in 96-well plates. To ensure feasible and cost-effective diversification of the library, we will use lentiviral transduction of, on average, one virus per cell, and selection for genomic integration followed by clonal expansion in the microplates. To ensure that drug exposure only lasts 24 hours, peptides will only be expressed upon doxycycline (Dox) exposure. After Dox induction we will expose the cells to cold (6 hrs, 32 C), and measure thermogenesis via UCP1 activation using mito-stress assay (seahorse analyzer). As a secondary endpoint, a control plate in 37 C will also be used to identify peptides that show cold-dependent, rather than constitutive, UCP1 activation for improved safety of thermogenesis. The positive control for UCP1 expression will be exposure to IL6, and negative control to vehicle. The top 5 clones will be diversified using error-prone PCR and subjected to further rounds of evolution, such as for a total of 4.

Selection for therapeutics reversing cold adaptation—One goal of this study and work is to generate therapeutics that induce a stable, but reversible, cold adaptation. After the drugs are selected and validated in vivo, we will induce cold-adaptation with a selected MAPS peptide and screen to find drugs that can reverse that adaptation.

Low throughput validation of the selected peptides in vitro—To validate the peptides obtained in the screen we will first perform in vitro experiments for UCP1 activation after cold exposure (6 hrs, 32C) and in larger numbers of cells (10⁶ cells) in biological quadruplicate. The top 5 most thermogenic peptides from the screen will be tested against known thermo-adaptive proteins (IL6, Bmp8b, Fgf21, Bmp7) and a negative control (GFP).

### In Vivo Screening for Thermo-Adaptive Therapeutics:

Delivery of the library into the tissues—The DNA library encoding peptides will be packaged in AAV8, which efficiently transduces BAT, and other NST-generating tissues. The library will also include small number (5%) of control genes: an intracellular GFP (negative control), and a membrane-tethered (see FIG. 12) IL6 (positive control) which will be validated in low throughput before the screening. A secondary positive control will be BAT brown adipocytes from mice adapted to cold by training. These controls will allow us to benchmark our peptides during single cell sequencing and evaluate noise levels in the screen. Each viral vector will also carry a fluorescent protein (mCherry) to benchmark gene delivery. The library will be dosed to transduce one AAV per cell, to avoid multiple peptides being expressed in the same cell. Multiple peptides could exert opposing effects and mask positive effects of a candidate drug. We will inject the AAVs into supraclavicular and interscapular BAT, and as a backup strategy, WAT and muscles, since both can undergo NST and collecting these tissues adds little to no cost.

In vivo directed evolution of cold-adaptive therapeutics-After 2 weeks to allow for expression of the doxycycline-responsive reverse transactivator with the lowest available background, the peptide expression will be induced with doxycycline. Previous studies show that expression in vivo starts on day 5 post administration of doxycycline, after which we will allow for 24 hour long expression and transfer mice (C57BL6J, both male and female) from 30 C into cold environment (4 C) for 6 hours. Immediately afterwards, the mice will be euthanized by CO2 overdose, and their BAT, and other tissues will be collected for ex vivo analysis of thermogenicity. The top 10 most thermogenic peptides will then be mutated by error-prone PCR and screened again for a total of 4 rounds of screening. The last round of evolution will only include a top ˜1000 peptides from round 3, such that each peptide is expressed in multiple cells to allow for a statistical comparison of each peptide. That strategy allowed us to quantitatively compare efficacy of thousands of viral vectors using RNA-sequencing in our previous in vivo screen.

Single cell sequencing for selection of peptides—The tissues will be dissociated, peptide-transduced BAT adipocytes FACS-sorted, and processed for single-cell sequencing. The number of mice in each round will be chosen to record data from at least 10,000 brown adipocytes. Often very few (e.g., n=2) mice can be effective in an in vivo screen. Since brown adipocytes are challenging to isolate, we will start with n=4 mice per round but larger numbers are feasible and add little cost. We will evaluate the cold adaptation by measuring expression of thermogenic genes, including UCP1, and identify the thermogenic peptides by sequencing peptides' RNA in the same cells.

Screening to reverse the cold adaptation—Once the therapeutic that induces cold adaptation is validated, the MAPS screening can be modified to reverse the adaptation. In short, we will inject mice with a library of AAVs, administer the top cold-adaptive therapeutic or a vehicle for 24 h, induce the expression of adaptation-reversing peptide library, expose the mice to warm temperature (30 C for 6 h), and measure thermogenic gene transcription to identify peptides that bring it to baseline.

### In Vivo Validation of Thermoadaptive Therapeutics:

Measurement of core temperature in cold environment—Maintaining core body temperature is essential for an individual's comfort, focus, and survival. We will expose mice to cold (4 C) for 6 h and measure their core temperature using a rectal probe and shivering using electromyography. To measure the stability of adaptation we will test core temperature in the cold at 12, 24, and 72 h after drug injection. Mice will be monitored daily for adverse events and weight loss to test safety; tissues will be later analyzed by necropsy.

Measurement of dexterity after treatment—Dexterity and coordination are negatively impacted by cold. To measure the effects of our therapeutics on the behavioral impacts of cold we will perform two assays. First, a staircase reaching task for dexterity. No data exists regarding the effects of cold on mouse dexterity, possibly because it can be easily measured in humans. However, dexterity in the cold is a relevant endpoint for various occupations. Second will be a rotarod test for coordination, which is affected by hypothermia.

Measurement of cold preference—Cold-adaptation reduces aversion to cold. Consequently, a two-temperature preference test can quantify the cold adaptation and its changes in response to the therapeutics.

With the preceding in mind, use of MAPS to identify thermoadaptive drugs (TADs) may, in certain embodiments, be summarized as follows:

Generate TADs with MAPS—uses in vivo screening to find extracellular peptide drugs that activate/deactivate thermogenesis in brown adipose tissue, may be done as follows;
a) Generate random library of cell-surface-displayed peptides.
b) Deliver one vector encoding the peptide per one brown adipocyte in vivo, express the peptides transiently for 24 h.
c) Expose mice to cold and harvest tissues.
d) Perform single-cell analysis to find cells generate most heat.
e) Identify peptides expressed in these heat-generating cells.
f) Choose 10 peptides inducing most effective thermogenesis. Mutagenize each peptide, and re-screen to identify improved peptides. Repeat 4×.
g) Synthesize the extracellular portion of the most thermogenic peptides—these are the cold-adaptive drugs (CADs) to be tested.
h) Once CADs are validated in task 3, another MAPS screen is used to find drugs reversing the induced cold adaptation.
   In some embodiments, an additional screen round can be done to optimize safety, tissue specificity, or speed of action.

In a further aspect, a step of behavioral and physiological validation of TADs may be performed. In one such example, mice are provided drugs for 24h, exposed to cold, and their dexterity, coordination, core temperature, subjective cold sensation, and safety are compared to cold acclimation training.

### Risks and Mitigation:

Risk and Mitigation 1-Readout noise in scRNA is too high—It is possible that the thermogenic genes' activation readout will be noisy and provide erroneous readout. This risk is mitigated by: 1) performing multiple rounds of screening, where ‘hits’ from a screen are being readministered and validated in another animal; 2) final validation in low-throughput; 3) Introducing positive controls with known cold adaptive effects. 4) reducing the noise by decreasing the library size in each round, thus having multiple cells receiving the same peptide drug.

Risk and Mitigation 2-Too few cells will be analyzed for obtaining reliable biochemical data-BAT contains relatively few cells. MAP screening is scalable, so if too few cells are obtained, we can simply increase the number of mice used.

Risk and Mitigation 3—The library will not contain peptides that have thermoadaptive properties—It is possible that none of the randomized peptides will have thermoadaptive properties. We are aware of this risk as it is a common problem in high throughput screening. We have introduced a number of methods to mitigate this problem we: 1) mutate known thermoadaptive proteins (e.g. IL6) and screen for improvement over the baseline; 2) perform multiple rounds of evolution and validations, optimizing sub-optimal peptides. 3) we screen multiple ‘starting points’ concurrently.

Risk and Mitigation 4—It is possible that adipocytes will be too fragile for tissue homogenization and single-cell sequencing—If necessary, we will use single-nuclei RNA-sequencing, or transgenic mice which show UCP1-induced iRFP720 expression, sorting iRFP positive cells, and bulk RNA-seq.

Risk and Mitigation 5—Safety, specificity, or speed of action need improvement—In our experience with in vivo screening tools, evolution of molecules with a positive screen results in high specificity for the target. However, if needed, the drugs' safety and specificity can be further improved as follows: 1) The final library of the ˜1000 most thermogenic peptides can be re-screened in mice at 30 C to find drugs that only induce thermogenesis in the cold as a safeguard against hyperthermia. 2) The BAT-specificity can be ensured by screening the library via systemic delivery, and single-cell RNA-seq of peripheral organs. Peptides lacking significant effects on transcriptome in organs' cells (e.g. heart, liver, kidney) are likely to be BAT-specific and safer. 3) Increased speed of action can be achieved by lowering the time of MAPS peptide expression from 24 h to a shorter time; checking thermogenesis at a later timepoint can screen for higher stability.

Risk and Mitigation 6—In vivo screen will need a focused library-Mitigation may be achieved by performing an in vitro screen in parallel.

Risk and Mitigation 7—Single-cell analysis is too noisy-Mitigation may be achieved by using a bulk RNA-sequencing alternative pipeline or by increasing sequencing depth.

The technology discussed herein has the potential to identify peptide therapeutics and/or combination therapeutics in an expedited fashion by assessing phenotypic changes in disease models. This approach allows for the development of therapeutics that have the potential to treat conditions where there is limited understanding around targets of interest, MOAs, etc. Theoretically, this concept of mechanism-agnostic peptide screening could be applied to the development of treatments for numerous indications including, but not limited to on non-fatty alcoholic liver disease and non-alcoholic steatohepatitis.

In particular, in accordance with the presently described techniques a model system of HepG2 cells that mimics steatohepatitis has been developed. Additionally, EPCs that contain therapeutic candidates have been engineered to allow the peptides to interact with extracellular receptors. These EPCs have been tested with peptides that are known to reduce lipid accumulation or have been implicated in disease pathways for non-alcoholic steatohepatitis (NASH). Experimental results have confirmed that treatment of the steatotic HepG2 cells with EPCs featuring these peptides does, in fact, result in the reduction of lipids.

Ongoing work is directed to testing this method with a more complex population of steatotic HepG2 cells (e.g., a range of lipid accumulation, as opposed to very low vs. very high levels) to more closely mimic actual experimental data. Samples may be tagged with barcodes that can be analyzed using RNA extraction, cDNA synthesis, PCR amplification of fragments, and NGS, to determine the number of cells in each bin with a certain amount of accumulated lipids.

As noted herein a screening platform as described may be applied to a series of therapeutic peptide candidates. Classes of peptides that are considered promising, such as for reducing intracellular lipid accumulation, may be screened, such as but not limited to peptides that contain leucine and glycine in the intracellular lipid accumulation context.

With this in mind, an aim of the presently described techniques is to facilitate the use of high-throughput screening of diverse peptide libraries. Such approaches may focus on peptides that are between 7-9 residues, due to the average size of FDA-approved synthetic peptides and logistics (i.e., >9 amino acids results in a library that is greater than 20⁹). Top therapeutic candidates may then be assessed via in vitro and in vivo studies, such as in mice. Computational work to develop peptide fitness prediction models may also be performed.

With respect to other techniques, other screening techniques include siRNA screening, however the presently described techniques differ from such other techniques for various reasons including, but not limited to: (1) the use of peptides and proteins which can be turned into drugs; (2) the use of an innovative molecular design which tethers the protein/peptide candidates to the cell surface. This tethering allows for extracellular action and thus enables simpler administration of proteins into the interstitial tissues. Other techniques such as siRNA require cell and gene delivery; (3) The presently described screen is performed in a small piece of tissue in vivo, and thus accounts for variables not present in vitro. siRNA and other similar screens are typically performed in vitro.

With the preceding in mind, it may be appreciated that the presently described techniques may facilitate the identification and production of new drugs and therapeutic agents. In particular, the presently described approach omits the step of in vitro testing and provides lead compounds that work in animals from the start. Additionally, due to high-throughput nature of the screen, hypotheses do not need to be made regarding the mechanism of the disease. This mechanism-agnostic aspect allows potential drugs and therapeutic agents to be found regardless of the status of knowledge in the field about the disease. Considering that current drug target research shows low translation probabilities, being able to look for other targets while we screen is a significant advantage. In addition, the presently described screen can be performed in a very similar way for multiple different disorders, and production of peptide drugs is standardized. This will standardization and a reduction in the expertise needed to conduct drug development and drug production, leading to further increase in efficiency. In particular, the presently described techniques are particularly well-suited for identifying potential therapeutics for chronic diseases where the phenotype of single cells is affected.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for phenotype-driven drug discovery, comprising:
packaging a nucleic acid library in a vector to generate a vector library for gene delivery, wherein the nucleic acid library encodes randomized peptides;
applying the vector library to a cellular model at a threshold multiplicity of infection corresponding to substantially single vector transduction per cell; and
screening the transduced single cells for cell survivability or one or more phenotypes to identify one or more peptides of interest.

2. The method of claim 1, wherein the nucleic acid library comprises a plasmid library.

3. The method of claim 1, wherein the cellular model comprises a disease model.

4. The method of claim 1, further comprising providing a period of gene expression between applying the vector library and screening the transduced single cells.

5. The method of claim 1, wherein the one or more phenotypes comprise one or more of visual appearance, gene expression or changes in gene expression, metabolism or changes in metabolism, or changes identifiable at the single-cell level.

6. The method of claim 1, comprising re-screening the one or more peptides of interest to evaluate effectiveness as therapeutics.

7. The method of claim 6, comprising testing one or more peptides of interest in a low-throughput process.

8. The method of claim 1, wherein the one or more peptides of interest are expressed intracellularly.

9. The method of claim 1, wherein the one or more peptides of interest are expressed extracellularly.

10. The method of claim 1, comprising:
synthesizing an oligonucleotide pool by performing randomized in silico mutagenesis; and
generating the nucleic acid library using the nucleic acid sequences within the oligonucleotide pool.

11. The method of claim 1, wherein the randomized peptides correspond to a fully or partially randomized peptide library.

12. The method of claim 1, wherein the viral vector comprises an adeno-associated virus (AAV) capsid or lentiviral vector (LV).

13. The method of claim 1, wherein the threshold multiplicity of infection comprises 0.01 to 100 multiplicity of infection.

14. An engineered extracellular protein carrier, comprising:
a leader sequence;
a receptor structure; and
a therapeutic protein or peptide candidate.

15. The engineered extracellular protein carrier of claim 14, wherein the extracellular protein carrier comprises a genetically-encodable, cell-surface tetherable structure configured to link the therapeutic protein or peptide candidate to a surface of a cell, wherein the therapeutic protein or peptide candidate, when linked to the surface of the cell, interacts with extracellular receptors.

16. The engineered extracellular protein carrier of claim 14, wherein the leader sequence comprises an Ig-κ secretion leader sequence.

17. The engineered extracellular protein carrier of claim 14, wherein the receptor structure comprises a platelet-derived growth factor receptor β (PDGFR-β).

18. The engineered extracellular protein carrier of claim 14, further comprising a linker sequence.

19. The engineered extracellular protein carrier of claim 18, wherein the linker sequence comprises a glycine-serine linker sequence.

20. The engineered extracellular protein carrier of claim 14, further comprising a fluorescent protein label.
